(19) United States
(12) Patent Application Publication
(10) Pub. No.: US 2006/0266956 A1
(43) Pub. Date: Nov. 30, 2006

(54) Method of expanding mineral ores using microwave radiation

(76) Inventors: Vladislav Sklyarevich, Bensalem, PA (US); Mykhaylo Shevelev, Feasterville, PA (US)

Correspondence Address: FLOYD B. CAROTHERS;CAROTHERS AND CAROTHERS, 445 FORT PITT BOULEVARD, SUITE 500, PITTSBURGH, PA, 15219, US

(21) Appl. No.: 11/182,257

(22) Filed: Jul. 15, 2005

Related U.S. Application Data
(60) Provisional application No. 60/684,806, filed on May 25, 2005.

(51) Int. Cl. G21G 5/00; A61N 5/00

(52) U.S. Cl. 250/492.1

(57) ABSTRACT

Here is described a method of expanding mineral ore particles, preferably perlite, to effect rapid heating of particles to a required softening temperature so that the selected size and shape of expanded ore particles is obtained. A microwave radiation with appropriate frequency and power density is chosen so as to accomplish particle heating to a required (e.g., softening) temperature in a selected short time while ensuring that the entire particle material is expanded at once. The method provides high efficiency of the expanding process that can be close to 100%.

### FIELD OF THE INVENTION

The invention relates to a method of processing expandable ores such as vermiculite, perlite and the other natural glass-like materials. More particularly, it relates to an improved method of producing finely divided particles, which are useful in filter and filler applications.

The present invention also can be applied to the thermal processing of non-expandable ores.

### BACKGROUND OF THE INVENTION

Millions of tons of expanded ores are produced annually worldwide. Mainly this is expanded perlite. It is widely used in dozens of areas for filtration, horticulture, high temperature insulation, coatings, and the like, as well as, filters for construction materials, ceiling tiles and the like.

Expanded ores, such as hydrous silicates of various metals, perlite, and the like, possess combined water. When, for example, granulated raw perlite is heated up to around 800-1100° C., the combined water (2% to 6%) present in the mineral structure vaporizes and the formed steam acts to expand the softened material, increasing the porosity of the structure and decreasing the original density. Thus, perlite can expand 10 to 15 times its original volume. Representative values of (bulk) density for crude perlite are 2200-2400 Kg/m³, for crushed perlite are 900-1100 Kg/m³ and for expanded (loose bulk) perlite, 60-120 Kg/m³.

Processes and apparatus for expanding mineral ores such as perlite are well known. The basic method was developed almost 50 years ago (see, for example, U.S. Pat. Nos. 2,853,241; 2,898,303) and there have been no principle changes made since that time. Typically, crushed, dried and sized expandable ore is heated in an open flame of liquid fuel or natural gas in horizontal (rotary, stationary), or most widely in vertical furnaces (see for example U.S. Pat. Nos. 2,853,241; 2,898,303; 4,318,691; 4,512,736; 4,521,182; 5,002,696; 4,525,388: 5,908,561; 6,712,898).

Such an approach yields a very low efficiency of the expanding process because ore particles are spread in a large volume and, in fact, the mix of air and particles is heated. The particle portion in this mix is minor. Therefore the actual efficiency of existing manufacturing processes usually does not exceed 20% in the best case.

Another problem with the existing method is the limit in particle size (density) because of the limit in heating rate that can be realistically achieved by using gas or fuel. Heat density for this heat source is determined mainly by temperature, which is constant. At the same time, the low density characteristic of expanded ore particles, for example, perlite, as well known, is very desirable for all applications.

The nature of this type of heating also limits product quality. Hot gas or fuel (as well as infrared) heats the perlite particle from the surface inward. The rest of the particle achieves heat through thermal conductivity and this requires some time. Therefore the expansion process starts from the surface layer and then moves inside. As was shown in Founti, M. and Klipfel, A.: Experimental and computational investigations of Nearly Dense Two-Phase Sudden Expansion Flows, Experimental Thermal and Fluid Science, Elsevier Science Publishers 17 (1998) pp. 27-36, the viscosity of perlite that has lost water rises by almost ten times (see FIG. 1). This means that the expansion of each following layer becomes more difficult, and this limits the size of the expanded particle.

Thus, there is a clear need in the art for a method for the rapid heating of expandable ores which allows increased product quality (reduced density of expanded particles) and increased efficiency of this process.

### SUMMARY OF THE INVENTION

According to the present invention, a method is provided for expanding mineral ore particles comprising continuous movement of the bulk of ore particles and exposing them to concentrated microwave radiation. Said radiation has a frequency between about 20 GHz to about 200 GHz. By selecting sufficient power density, the particles are heated to their required softening temperature in a selected time and, as a result, the selected size and shape of expanded ore particles is obtained. The yield of the process can be increased if the thickness of the bulk is selected to be less than the skin layer for the used microwave frequency in the bulk.

The method provides the size selection of particles by conveying them out continuously and independently.

### BRIEF DESCRIPTION OF THE DRAWINGS

FIG. 1 graphically illustrates prior art investigation of perlite viscosity depending on water content.

FIG. 2 is a schematic view illustrating the basic gyrotron beam installation used in the inventive method.

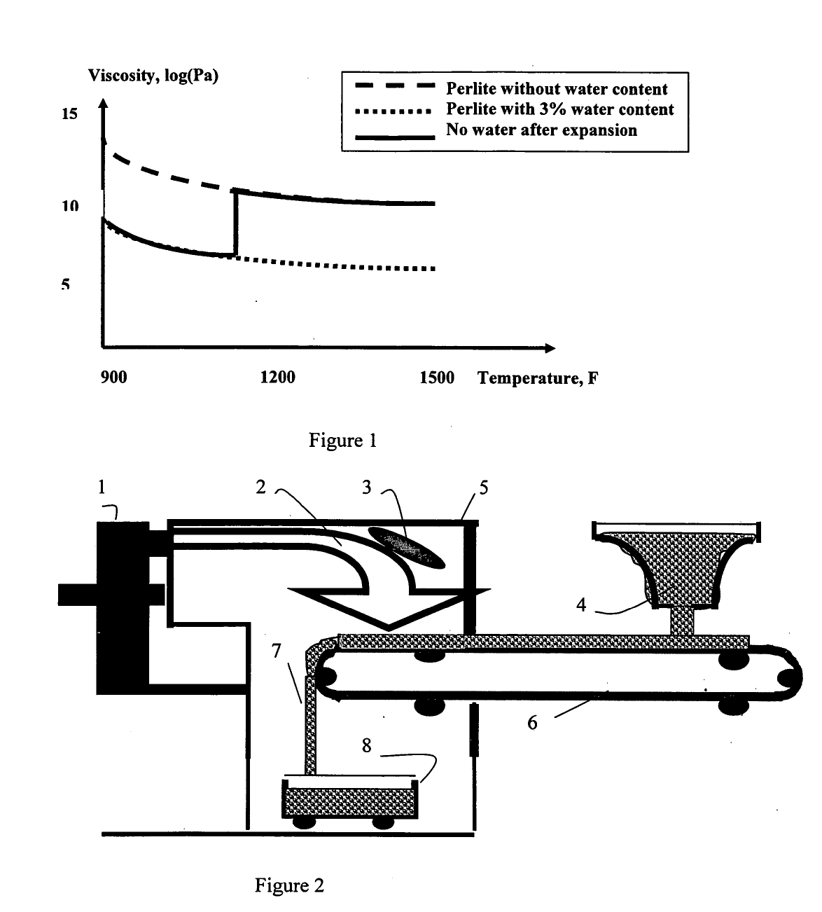

### DETAILED DESCRIPTION OF THE INVENTION

The present invention relates to a method of processing expandable ores such as vermiculite, perlite and other natural glass-like materials. More particularly, it relates to an improved method of producing finely divided particles, which are useful in filter and filler applications.

In the inventive method, a bulk of ore particles continuously moves and is exposed to concentrated microwave radiation with a frequency between about 20 GHz to about 200 GHz and a sufficient power density. The microwave generators at the chosen frequency are available at power levels of dozens, and even hundreds of kilowatts CW, for example gyrotrons, klystrons, traveling wave tubes, backward wave oscillators and others.

The process parameters are chosen so as to accomplish heating ore particles volumetrically and in a time sufficient to bring particle material to a softening point while water remains inside (heating rate up to 1000 C per second and more). This allows achieving greater expansion because the material viscosity with water is less than when water escapes from inside. By selecting the power density and processing time, the desirable size of expanded particles can be produced.

In the embodiments of the invention discussed above, the thickness of the ore particle bulk is selected to be less than the skin layer for the used microwave frequency in the bulk. In this case the coupling of microwave energy by ore particles is the highest, and close to 100%. All particles are heated at about the same rate.

Because of high power density, the escaped water creates a high thrust force that moves the expanded particles from the bulk. They can be collected and separated by sizes by conveying them out continuously and independently using, for example a few conveyers or collectors located at different distances from the conveyer that carries the non-expanded ore particles.

In the embodiments of the invention discussed above, particles can be separated by moistening them before exposure to the microwave. The microwave high power density creates steam pressure that makes the particles move outside the bulk in the area where the concentrated microwave is. The particles are heated during this movement and accumulate at different distances from the bulk, correspondent to size.

The capital cost of the invented method can be reduced if particles are preheated before processing by concentrated microwave radiation, but not higher than to around a temperature when expandable ore loses water.

The inventive method is generally applicable to the thermal treatment of any expandable or non expandable ore material and for producing expanded materials for any filtration and filler applications and the like. It saves energy, production and capital costs, and increases quality of products.

The present invention can also be applied for rapid heating of perlite-like materials, for example, diamateous earth (DE) in the process of straight or flux calcining. Rapid heating of dried DE by microwave makes the conditions for aggregating treated material without forming crystalline silica (microwave enhanced sintering). For example, non organic material with higher absorption to the applied microwave, may be added to the DE. This non-organic material serves as a binder and provides aggregating at temperatures less than the temperature of crystalline silica formation.

The present invention can also be applied for removing organic components from ore powder. Most organic materials have higher absorption to the microwave than ore powder. Under rapid and high power density microwave exposure, the organic components heat up and volatilize faster than perlite reaches critical temperature. Under the right set up, the efficiency of this process may be around 100%.

The present invention has been described in an illustrative manner. It is to be understood that the terminology that has been used is intended to be in the nature of words of description rather than of limitation. Many modifications and variations of the present invention are possible in light of the above teachings. Therefore, within the scope of the appended claims, the present invention may be practiced other than as specifically described.

### Microwave Source and Irradiation Set-Up

Microwave radiation with the necessary frequency and power density can be achieved using generators such as the gyrotron, klystron, and the like. In FIG. 2 an example of a microwave installation that can be used in the inventive method is illustrated.

The microwave unit consists of a gyrotron 1 that operates at a frequency of 82.9 GHz (wavelength λ≈3.62 mm) with a maximum output CW power of 15 kilowatts. The concentrated microwave radiation (the gyrotron beam) 2 that is generated by the gyrotron 1 is directed by a mirror 3. The mirror 3 forms the necessary shape and distribution of microwave power in the beam 2 and directs it to the perlite 4. Perlite 4 is continuously delivered to microwave chamber 5 by a conveyer 6. The microwave beam heats perlite 4 and expanded material 7 flows into the carrier 8.

It is understood that the particular installation illustrated in FIG. 2 is optimally designed for research and development or demonstration testing of the inventive method. A person of ordinary skill in the art can easily modify the installation for manufacturing processes of various scales. Using this setup, it is possible to perform rapid heating of the perlite to expand it.

### Exemplary Determination of Process Parameters

The following example is presented to provide a more detailed explanation of the present invention and of the preferred embodiments thereof and is intended as illustration and not limitation.

A non expanded perlite powder was chosen for the heating experiments. Perlite was sprinkled onto a conveyer with a thickness that was determined as the skin layer of the used microwave in perlite. This was 20 mm for microwave frequency 83 GHz. The power density was around 5 kW per cm2 with the total power around 15 kW and production speed of 30 gr/sec. Heating rate was around 2,000 C/sec. Based on this data, the efficiency of the process was estimated as 97%. The volume and weight of expanded perlite was measured and its density was calculated. It was less than 45 Kg/m³.

What is claimed is:

1. A method of expanding mineral ore particles comprising continuously moving a bulk of ore particles while exposing them to concentrated microwave radiation having a frequency between about 20 GHz to about 200 GHz and sufficient power density to heat the particles to their required softening temperature in a selected time whereby a selected size and shape of expanded ore particles is obtained.

2. The method of claim 1 wherein the thickness of the bulk is selected to be less than the skin layer for the used microwave frequency in the bulk.

3. The method of claim 1 wherein the expanded particles are distributed according to size by conveying them out continuously and independently.

4. The method of claim 1 wherein the mineral ore particles are preheated before exposure to concentrated microwave radiation but not higher than the temperature under which said ore particles loose water.
